(19) United States
(12) Patent Application Publication
(10) Pub. No.: US 2023/0338142 A1
(43) Pub. Date: Oct. 26, 2023

(54) STEERABLE DELIVERY DEVICES AND SYSTEMS FOR A STENTED PROSTHESIS

(71) Applicant: Medtronic Vascular, Inc., Santa Rosa, CA (US)

(72) Inventor: Padraigh JENNINGS, Galway (IE)

(21) Appl. No.: 17/922,368

(22) PCT Filed: May 21, 2021
(86) PCT No.: PCT/US2021/033542

§ 371 Date: Oct. 30, 2022

Related U.S. Application Data
(60) Provisional application No. 63/028,322, filed on May 21, 2020.

(51) Int. Cl. A61F 2/24; A61F 2/95; A61F 2/966

(52) U.S. Cl. CPC A61F 2/2436; A61F 2/9517; A61F 2/2418; A61F 2/966; A61F 2002/9665

(57) ABSTRACT

A stented prosthesis delivery device with steering capabilities. One or more actuation bodies (e.g., shape memory polymer) are located at a distal region of the delivery device, and are selectively stimulated by a user (e.g., via a controller or joy stick at the device's handle) to effect desired steering.

### BACKGROUND

The present disclosure relates to delivery devices for implanting a stented prosthesis, such as a transcatheter stented prosthetic heart valve. More particularly, it relates to catheter-based devices with steering capabilities for implanting a stented prosthetic heart valve or other stented prosthesis.

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of the valve prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery, and expanded upon deployment at or within the native valve. One type of valve stent can be initially provided in an expanded or uncrimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath or catheter for transluminal delivery. Retracting the sheath from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place. One example of a stented prosthetic valve is disclosed in U.S. Pat. No. 5,957,949 to Leonhardt et al., which is incorporated by reference herein in its entirety.

Regardless of the actual shape and configuration of the transcatheter prosthetic heart valve, the catheter-based device or system for delivering the prosthesis are required to track a patient's anatomy to a desired location within the vasculature. As a point of reference, the preferred delivery approach oftentimes includes one or more significant bends or turns. In many instances, the native anatomy creates the “tight” or small radius of curvature bends. A retrograde approach to the aortic valve is but one example.

As implied by the above, a stented prosthesis delivery device is often required to traverse tortuous and tight anatomical tracks as part of the delivery procedure. By design, the components(s) of the delivery device (e.g., catheter) traversing the vasculature must have sufficient column strength to transfer a pushing force applied at a proximal side or section of the device into forward motion at a distal side or section within the vasculature, while providing sufficient flexibility to navigate the anatomy without imparting undue stress on the vasculature. These performance requirements are contrasting by nature, and it can be difficult to achieve for many stented prosthesis delivery device designs. For example, delivery catheter systems typically employed for trans-femoral aortic valve implant (“TAVI”) of a self-expanding stented prosthetic heart valve can be characterized as having a large profile and stiff shaft relative to the intended anatomical track. While existing TAVI delivery catheter systems are well-accepted for most patients, the size and/or stiffness characteristics may give rise to concerns in some instances. For example, when traversing the tortuous and tight anatomical track, the catheter/capsule (or other components of the delivery device) inherently comes into contact with the native anatomy. If the delivery device construction relies upon the native anatomy to naturally assist or “force” the catheter to the shape of the anatomical track, a stiffness and/or size of the delivery device catheter may negatively stress contacted anatomy. These concerns can be more prevalent with a patient having an anatomy that is more tortuous or frail than in the nominal case. Similar concerns can arise with patient vasculatures containing large pieces of calcium. If dislodged or disturbed, the calcium deposit may form an embolism and subsequent clot.

Although there have been multiple advances in transcatheter prosthetic heart valves and related delivery systems and techniques, there is a continuing need to provide different delivery tools for controlled delivery of the prosthesis to the native valve site.

### SUMMARY

Some aspects of the present disclosure are directed toward a delivery device for implanting a stented prosthesis, for example a stented prosthetic heart valve. The delivery device includes a handle assembly, an outer shaft, an inner shaft, at least one actuation body, and a controller. The outer shaft extends from the handle assembly. The inner shaft is disposed within the outer shaft. The actuation body is carried by the outer shaft. The controller is carried by the handle assembly and is connected to the actuation body for selectively prompting delivery of stimulation to the actuation body. The delivery device is configured to provide a loaded state in which a stented prosthesis, such as a stented prosthetic heart valve, is compressed over the inner shaft and retained within a capsule of the outer shaft. The actuation body is operable to deflect the outer shaft in response to the delivered stimulation. In some embodiments, the actuation body includes or comprises a shape memory polymer, and the delivered stimulation is an electrical current. In other embodiments, four actuation bodies are provided, equidistantly spaced about a circumference of the outer shaft at a distal region thereof.

### BRIEF DESCRIPTION OF THE DRAWINGS

FIG. 1 is a simplified perspective view of a delivery device in accordance with principles of the present disclosure;

FIG. 2 is a simplified side view of the delivery device of FIG. 1 loaded with a prosthetic heart valve;

FIG. 3A is a cross-sectional view of the delivery device of FIG. 1, taken along the line 3A-3A;

FIG. 3B is a cross-sectional view of the delivery device of FIG. 1, taken along the line 3B-3B;

FIG. 4A is a perspective view of an actuation body useful with the delivery device of FIG. 1 in an un-activated state;

FIG. 4B is a perspective view of the actuation body of FIG. 4A in an activated state;

FIG. 5 schematically illustrates a deflection assembly useful with the delivery device of FIG. 1;

FIG. 6A is an enlarged, perspective view of a user control device useful with the delivery device of FIG. 1;

FIG. 6B is a top plan view of the user control device of FIG. 6A;

FIG. 7A is a side view of a stented prosthetic heart valve useful with the systems, devices and methods of the present disclosure and in a normal, expanded condition; and

FIG. 7B is a side view of the prosthetic heart valve of FIG. 7A in a compressed condition.

### DETAILED DESCRIPTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. As used herein with reference to an implanted valve prosthesis, the terms “distal”, “outlet”, and “outflow” are understood to mean downstream to the direction of blood flow, and the terms “proximal”, “inlet”, or “inflow” are understood to mean upstream to the direction of blood flow.

As referred to herein, stented transcatheter prosthetic heart valves useful with and/or as part of the various systems, devices and methods of the present disclosure may assume a wide variety of different configurations, such as a bioprosthetic heart valve having tissue leaflets or a synthetic heart valve having polymeric, metallic or tissue-engineered leaflets, and can be specifically configured for replacing any of the four valves of the human heart. Thus, the stented prosthetic heart valve useful with the systems, devices, and methods of the present disclosure can be generally used for replacement of a native aortic, mitral, pulmonic or tricuspid valve, or to replace a failed bioprosthesis, such as in the area of an aortic valve or mitral valve, for example. In yet other embodiments, the devices, systems, and methods of the present disclosure can be useful for delivering other stented prostheses that may or may not be a stented prosthetic heart valve.

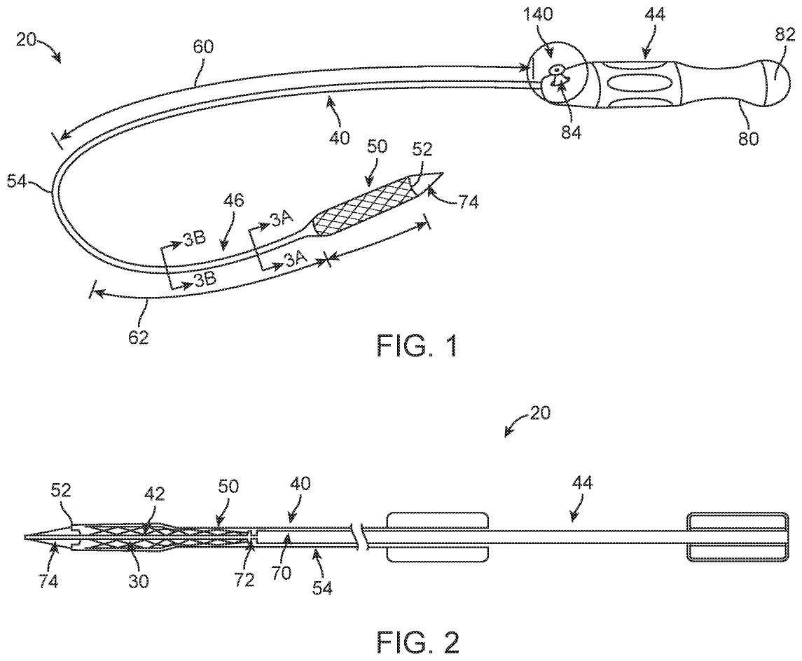

One example of a delivery device 20 in accordance with principles of the present disclosure is provided in FIG. 1. The delivery device 20 is further illustrated in simplified form in FIG. 2, and loaded with a stented prosthesis 30, such as a stented prosthetic heart valve. The delivery device (or delivery catheter system) 20 includes an outer shaft assembly 40, an inner shaft assembly 42, a handle assembly 44, and a deflection assembly 46 (referenced generally) including one or more actuation bodies (hidden in the view of FIG. 1, but described in greater detail below). Details on the various components are provided below. In general terms, however, the delivery device 20 combines with a stented prosthesis, such as the stented prosthetic heart valve 30, to form a system for performing a therapeutic procedure on a patient, such as a therapeutic procedure on a defective heart valve of a patient. The delivery device 20 provides a loaded or delivery state (shown in FIGS. 1 and 2) in which the stented prosthesis 30 is loaded over the inner shaft assembly 42 and is compressively retained within a capsule 50 of the outer shaft assembly 40. The outer shaft assembly 40 can be manipulated to withdraw the capsule 50 proximally from over the prosthetic heart valve 30 via operation of the handle assembly 44, permitting the prosthesis 30 to self-expand and release from the inner shaft assembly 42. The delivery device 20 can optionally include other components that assist or facilitate or control complete deployment. Regardless, the deflection assembly 46 is operable to deflect or bend the corresponding segment of the outer shaft assembly 40 in controlled fashion, effectuating a change in spatial orientation or shape delivery device 20 at which the actuation body is located, to effect steering of the corresponding segment, for example via operation of a controller carried by the handle assembly 44.

Various features of the components 40-44 reflected in FIGS. 1 and 2 can be modified or replaced with differing structures and/or mechanisms. Thus, the present disclosure is in no way limited to the outer shaft assembly 40, the inner shaft assembly 42, or the handle assembly 44 as shown and described below. Any construction that generally facilitates compressed loading of a stented prosthesis, such as a stented prosthetic heart valve, over an inner shaft via a retractable outer sheath or capsule is acceptable. Further, the delivery device 20 can optionally include additional components or features not shown.

In some embodiments, the outer shaft assembly 40 extends from the handle assembly 44 to a distal end 52, and includes the capsule 50 and an outer shaft 54. The outer shaft assembly 40 can be akin to a catheter, defining a lumen that extends from the distal end 52 through the capsule 50 and at least a portion of the outer shaft 54. The capsule 50 extends distally from the outer shaft 54, and in some embodiments has a more stiffened construction (as compared to a stiffness of the outer shaft 54) that exhibits sufficient radial or circumferential rigidity to overtly resist the expected expansive forces of the stented prosthetic heart valve 30 when compressed within the capsule 50. For example, the outer shaft 54 can be a polymer tube embedded with a metal braiding, whereas the capsule 50 includes a laser-cut metal tube that is optionally embedded within a polymer covering. Alternatively, the capsule 50 and the out shaft 54 can have a more uniform or even homogenous construction (e.g., a continuous polymer tube). Regardless, the capsule 50 is constructed to compressively retain the stented prosthetic heart valve 30 (or other stented prosthesis) at a predetermined diameter when loaded within the capsule 50, and the outer shaft 54 serves to connect the capsule 50 with the handle assembly 44. The outer shaft 54 (as well as the capsule 50) is constructed to be sufficiently flexible for passage through a patient's vasculature, yet exhibits sufficient longitudinal rigidity to effectuate desired axial movement of the capsule 50. In other words, proximal retraction of the outer shaft 54 is directly transferred to the capsule 50 and causes a corresponding proximal retraction of the capsule 50. In other embodiments, the outer shaft 54 is further configured to transmit a rotational force or movement onto the capsule 50.

Regardless of an exact construction, the outer shaft 54 can be viewed as having or defining a proximal region 60 and a distal region 62. The proximal region 60 extends from the handle assembly 44. The distal region 62 is opposite the proximal region 60 immediately adjacent the capsule 50. The distal region 62 can be considered a segment of the outer shaft 54 immediately proximal, but not including, the capsule 50 (e.g., the distal region 62 does not encompass or cover the prosthetic heart valve 30 in the loaded state of FIGS. 1 and 2).

The inner shaft assembly 42 can have various constructions appropriate for supporting the outer shaft assembly 40, including supporting the prosthetic heart valve 30 disposed thereon relative to the capsule 50. In some embodiments, the inner shaft assembly 44 includes an inner shaft 70 (i.e., a singular, continuous tubular shaft; two or more differently constructed tubular shafts that are connected to one another; etc.). Regardless, the inner shaft assembly 44 forms or defines at least one lumen (not shown) sized, for example, to slidably receive a guide wire (not shown).

The inner shaft assembly 42 further includes, or is connected to or includes a valve retainer or mechanism 72 and a tip 74. The valve retainer 72 can assume various forms and is configured to selectively capture or retain corresponding feature of the prosthetic heart valve 30 (thus retaining the prosthetic heart valve 30 relative to the inner shaft assembly 42 in the loaded state). In some non-limiting embodiments, for example, the valve retainer 72 includes one or more fingers sized to be received within corresponding apertures formed by a stent or frame of the prosthetic heart valve 30. Alternatively or in addition, the valve retainer 72 can be configured to selectively receive a corresponding feature (e.g., posts) provided with the prosthetic heart valve 30. When the capsule 50 is retracted proximally beyond the valve retainer 72, the stented prosthetic heart valve 30 can completely release or deploy from the delivery device 20. The delivery device 70 can optionally include other components that assist or facilitate or control complete deployment. The tip 74 forms or defines a nose cone having a distally tapering outer surface adapted to promote atraumatic contact with bodily tissue. The tip 74 can be fixed or slidable relative to the inner shaft 70.

The handle assembly 44 generally includes a housing 80 and one or more deployment actuator mechanisms (i.e., controls) 82 (referenced generally). The housing 80 can have any shape or size appropriate for convenient handling by a user. The housing 80 maintains the actuator mechanism(s) 82, with the handle assembly 44 configured to facilitate sliding movement of the outer shaft assembly 40 relative to the inner shaft assembly 42 via operation of the deployment actuator mechanism(s) 82. In addition, the handle assembly 44 includes a user control device 84 carried by the housing 80 as described in greater detail below.

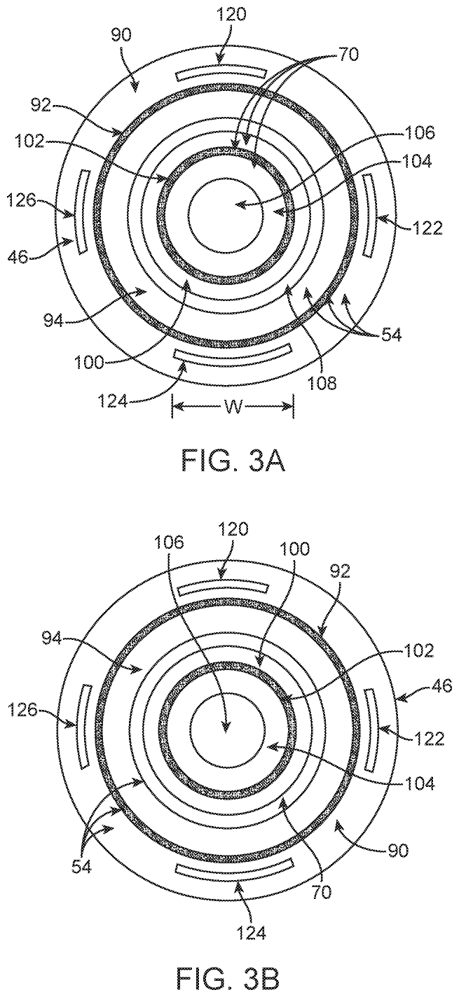

With the above general explanations of exemplary embodiments of the components 40-44 in mind, portions of one embodiment of the deflection assembly 46 is shown in greater detail in FIGS. 3A and 3B. As a point of reference, the cross-sectional views of FIGS. 3A and 3B are taken along the distal region 62 of the outer shaft 54, and reflect and that in some non-limiting embodiments, the outer shaft 54 can have a multi-layer construction, including, for example, a jacket layer 90, a braid layer 92, and a liner layer 94. Similarly, the inner shaft 70 is shown as optionally having a multi-layer construction including a jacket layer 100, a braid layer 102, and a liner layer 104. As described above, the inner shaft 70 can define a lumen 106 (e.g., for slidably receiving a guide wire). Finally, a gap 108 can exist between the outer and inner shafts 54, 70.

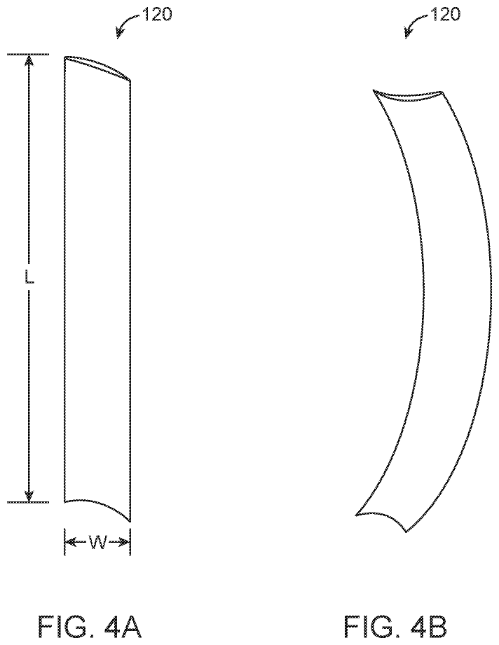

In some embodiments, the deflection assembly 46 includes one or more actuation bodies, such as a first actuation body 120, a second actuation body 122, a third actuation body 124, and a fourth actuation body 126. The actuation bodies 120-126 can be identical, such that the following description of the first actuation body 120 applies equally to the second-fourth actuation bodies 126. The actuation body 120 is generally configured to change shape in response to an applied stimulation. In some embodiments, the actuation body 120 is comprised of or includes a shape memory polymer (SMP) exhibiting the ability to return from a deformed state or shape to an original shape induced by an external stimulus, such as an electrical current, thermal/temperature change, magnetic field, or light. As understood by one of ordinary skill in the art, SMPs include thermoplastic and thermoset (covalently cross-linked) polymeric materials. In some non-limiting examples, the SMP material is an electro-active or conducting SMP composite with carbon nanotubes, short carbon fibers, carbon black, metallic Ni powder, etc., and are responsive or “triggered” by electricity. Regardless, a shape of the actuation body 120 in an un-activated state (i.e., in which no stimulus is being applied) is shown in FIG. 4A. Activation of the actuation body 120 cases the actuation body 120 to revert or self-transition from the shape of FIG. 4A to a pre-shaped form, for example the shape of FIG. 4B that otherwise exhibits a bend.

In some embodiments, the actuation body 120 has an elongated shape, with a length L greater than a width W (identified in FIG. 4A). Upon final assembly, and with additional reference to FIGS. 3A and 3B, the actuation body 120 is arranged relative to the outer shaft 54 such that the length L extends along, or parallel to, a longitudinal axis defined by the outer shaft 54, and the width W extends along a circumference of the outer shaft 54 in some embodiments. Where provided, the second—fourth actuation bodies 122-126 are similarly arranged, and can be substantially equidistantly spaced (i.e., within 10% of a truly equidistant spacing) about the circumference of the outer shaft 54. Other arrangements are also acceptable. Further, while four of the actuation bodies 120-126 are shown, any other number, either greater or lesser, is also acceptable.

As reflected by a comparison of FIGS. 1, 3A, and 3B, the actuation bodies 120-126 are located along the distal region 62 of the outer shaft 54, and terminate immediately proximal the capsule 50. For example, and with reference to the cross-sectional planes identified in FIG. 1, the actuation bodies 120-126 extend from a location approximately at the cross-section line 3A-3A to a location approximately at the cross-sectional line 3B-3B. With this arrangement, a change in shape at one (or more) of the actuation bodies 120-126 will effect a bend in the delivery device 20 along the distal region 62, serving to steer the capsule 50 (and the prosthetic heart valve 30 contained therein) with minimal force or impact on the proximal region 60.

The actuation bodies 120-126 can be assembled to one or more other components of the delivery device 20, for example the outer shaft 54, in various fashions. In some non-limiting examples, the actuation bodies 120-126 can be embedded into a thickness of the outer shaft 54, for example within the jacket layer 90 (e.g., a polymer layer). Alternatively, the actuation bodies 120-126 can be embedded within a different layer of the outer shaft 54, can be fastened to an exterior of the outer shaft 54, can be fastened to another component of the delivery device 20 other than the outer shaft 54, etc.

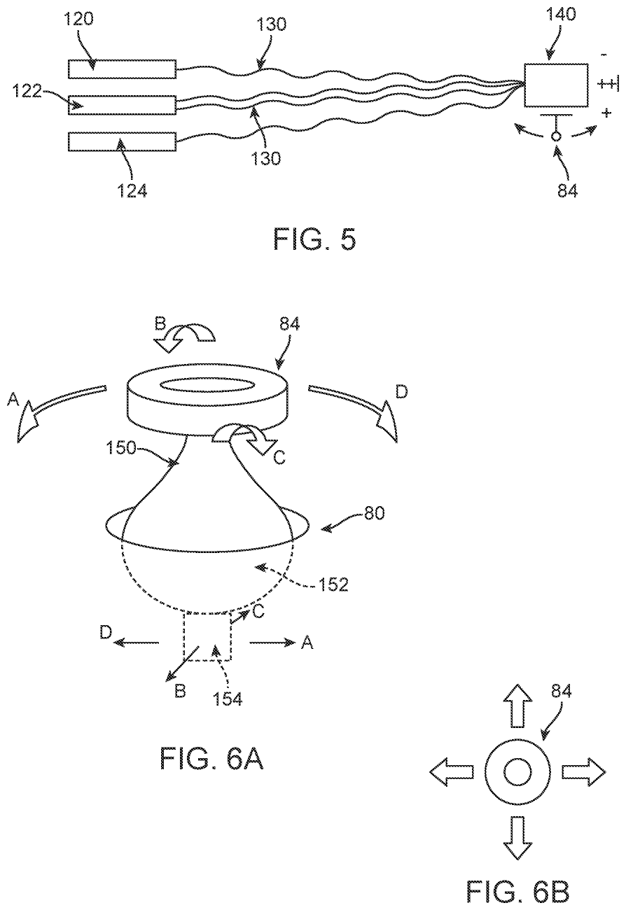

With embodiments in which the actuation body or bodies 120-126 include or comprise a shape memory polymer, the deflection assembly 46 can further include one or more components configured to deliver a stimulus to the actuation bodies 120-126. For example, FIG. 5 is a simplified representation of three of the actuation bodies 120-124 (the fourth actuation body 126 is hidden in the view). A wire 130 extends from each of the actuation bodies 120-126 to a controller 140. With additional reference to FIG. 1, the controller 140 (referenced generally in FIG. 1) can be located at or provided with the handle assembly 44, with the wires 130 extending along the outer shaft assembly 40 to the handle assembly 44. An electrical contact (not shown) for each of the wires 130 is provided at the controller 140 (e.g., a separate electrical contact can be provided for each individual wire 130, or two (or more) of the wires 130 can be connected to a single electrical contact). The controller 140, in turn, is connected to or includes a power source (not shown), and includes the user control device 84 that is operable to selectively complete an electrical connection between the power source and respective ones of the electrical contacts.

The control device 84 can assume various forms, and in some embodiments can include or comprise a joy stick or joy stick-like construction. One non-limiting example of the control device 84 is shown in FIG. 6A, along with a portion of the handle assembly housing 80. The control device 84 is carried by and extends from a surface of the housing 80, and is retained thereto so as to be pivotable or translatable in at least four directions (identified, for example, in FIG. 6B). The control device 84 can include a head 150, a base 152 and a contact arm 154. The base 152 is rotatably secured to the housing 80, with the head 150 projecting outwardly from the housing 80 for a user access. The contact arm 154 is disposed within the housing 80, and arranged to selectively contact (and create an electrical connection to) the electrical contact(s) associate with the wires 130 (FIG. 5). With this one, non-limiting construction, a user can manipulate the head 150 to selectively deliver stimulation to selected ones of the actuation bodies 120-126 (FIG. 3) via the contact arm 154, effecting dual direction, dual action motion control over bending or steering at the distal region 62 (FIG. 1). Stated otherwise, the control device 84 serves as a dual direction, dual axis control stick that is used to control the steering function provided by the deflection assembly 46. With reference between FIGS. 1, 3A, and 6, operation of the deflection assembly 46 includes moving the control device 84 in a direction or combination of directions to send current to a desired actuation body 120-126 otherwise located in the distal region 62; where the actuation bodies 120-126 include or comprise a shape memory polymer, the current in the actuation body or bodies 120-126 will cause activation of the shape memory polymer and the subsequent deflection of the distal region 62 in that direction. In some embodiments, the deflection assembly 46 will allow a user to deflect and steer the delivery device 20, and in particular the distal region 62, in the up or down direction at any one time, as well as right and left while in this downward or upward deflection. The ability to provide this active steering optionally in all directions can enable the delivery device 20 to, for example, follow a guidewire with minimal resistance and maximum efficiency to deliver the therapy (e.g., the prosthetic heart valve 30) to the intended site with minimal trauma to the patient.

As mentioned above, the delivery devices and systems of the present disclosure can be useful with the delivery of a stented prosthesis, such as a stented prosthetic heart valve. In general terms, the stented prosthetic heart valves of the present disclosure include a stent or stent frame having an internal one maintaining a valve structure (tissue or synthetic), with the stent frame having a normal, expanded condition or arrangement and collapsible to a compressed condition or arrangement for loading within a delivery device. The stent frame is normally constructed to self-deploy or self-expand when release from the delivery device. For example, the stents or stent frames are support structures that comprise a number of struts or wire segments arranged relative to each other to provide a desired compressibility and strength to the prosthetic heart valve. The struts or wire segments are arranged such that they are capable of self-transitioning from a compressed or collapsed condition to a normal, radially expanded condition. The struts or wire segments can be formed from a shape memory material, such as a nickel titanium alloy (e.g., Nitinol™). The stent frame can be laser-cut from a single piece of material, or can be assembled from a number of discrete components.

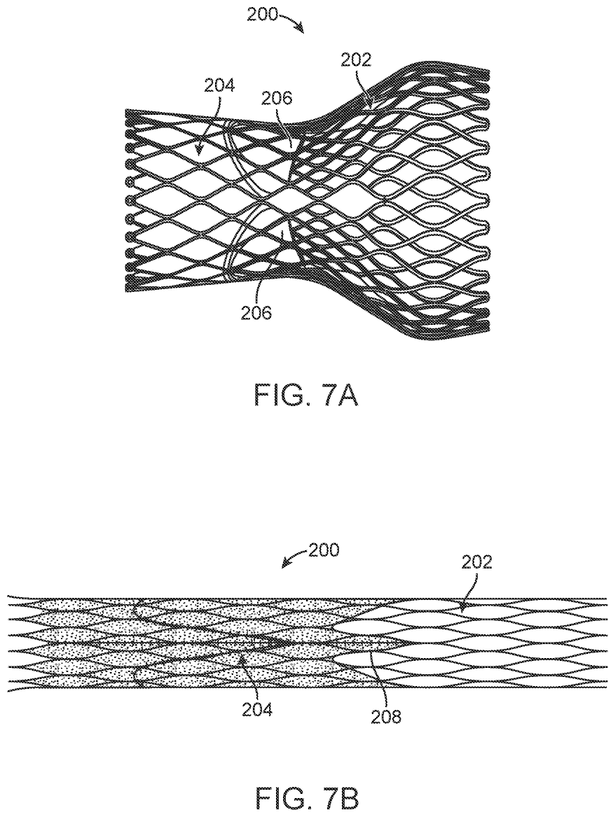

With the above understanding in mind, one simplified, non-limiting example of a stented prosthetic heart valve 200 useful with systems, devices and methods of the present disclosure is illustrated in FIG. 7A. As a point of reference, the prosthetic heart valve 200 is shown in a normal or expanded condition in the view of FIG. 7A; FIG. 7B illustrates the prosthetic heart valve 200 in a compressed condition (e.g., when compressively retained within an outer catheter or sheath as described below). The prosthetic heart valve 200 includes a stent or stent frame 202 and a valve structure 204. The stent frame 202 can assume any of the forms mentioned above, and is generally constructed so as to be self-expandable form the compressed condition (FIG. 7B) to the normal, expanded condition (FIG. 7A).

The valve structure 204 can assume a variety of forms, and can be formed, for example, from one or more biocompatible synthetic materials, synthetic polymers, autograft tissue, homograft tissue, xenograft tissue, or one or more other suitable materials. In some embodiments, the valve structure 204 can be formed, for example, from bovine, porcine, equine, ovine and/or other suitable animal tissues. In some embodiments, the valve structure 204 can be formed, for example, from heart valve tissue, pericardium, and/or other suitable tissue. In some embodiments, the valve structure 204 can include or form one or more leaflets 206. For example, the valve structure 204 can be in the form of a tri-leaflet bovine pericardium valve, a bi-leaflet valve, or another suitable valve. In some constructions, the valve structure 204 can comprise two or three leaflets that are fastened together at enlarged lateral end regions to form commissural joints 208, with the unattached edges forming coaptation edges of the valve structure 204. The leaflets 206 can be fastened to a skirt that in turn is attached to the frame 202.

With the one exemplary construction of FIGS. 7A and 7B, the prosthetic heart valve 200 can be configured (e.g., sized and shaped) for replacing or repairing an aortic valve. Alternatively, other shapes are also envisioned, adapted to mimic the specific anatomy of the valve to be repaired (e.g., stented prosthetic heart valves useful with the present disclosure can alternatively be shaped and/or sized for replacing a native mitral, pulmonic or tricuspid valve).

The delivery devices, systems and methods of the present disclosure provide a marked improvement over previous designs. By providing the delivery device with a robust deflection assembly, the delivery device can be readily operated to steer or effectuate desired bends or deflections commensurate with a desired delivery path presented by the anatomy of the particular procedure, even with more rigid or large outer sheath or catheter designs.

Although the present disclosure has been described with reference to preferred embodiments, workers skilled in the art will recognize that changes can be made in form and detail without departing from the spirit and scope of the present disclosure. For example, while the devices and systems of the present disclosure have been described as being useful for delivering a stented prosthetic heart valve, a number of other implantable devices can be employed.

1. A delivery device for implanting a stented prosthesis, the device comprising:
a handle assembly;
an outer shaft extending from the handle assembly;
an inner shaft disposed within the outer shaft;
a first actuation body carried by the outer shaft;
a controller carried by the handle assembly and connected to the first actuation body for selectively prompting delivery of stimulation to the first actuation device;
wherein the delivery device is configured to provide a loaded state in which a stented prosthesis is compressed over the inner shaft and retained within a capsule of the outer shaft;
and further wherein the first actuation body is operable to deflect the outer shaft in response to the delivered stimulation.

2. The delivery device of claim 1, wherein the first actuation body is a shape memory polymer.

3. The delivery device of claim 1, wherein the first actuation body is electrically connected to the controller.

4. (canceled)

5. The delivery device of claim 1, wherein the first actuation body is located at a distal region of the outer shaft.

6. (canceled)

7. The delivery device of claim 5, wherein the first actuation body has length greater than a width, and further wherein the first actuation body is arranged relative to the outer shaft such that the width extends along a circumference of the outer shaft.

8. The delivery device of claim 7, further comprising a second actuation body carried by the outer shaft and connected to the controller.

9. The delivery device of claim 8, wherein the second actuation body is located opposite the first actuation body relative to the circumference of the outer shaft.

10. The delivery device of claim 9, further comprising a third actuation body and a fourth actuation body both carried by the outer shaft and connected to the controller, wherein the first-fourth actuation bodies are equidistantly spaced from one another along the circumference of the outer shaft.

11. The delivery device of claim 10, wherein the controller includes a joy stick.

12. The delivery device of claim 1, wherein the delivery device is configured for implanting a stented prosthetic heart valve.

13. A system for performing a therapeutic procedure on a patient, the system comprising:
a delivery device comprising:
a handle assembly,
an outer shaft extending from the handle assembly and including a capsule,
an inner shaft disposed within the outer shaft,
a first actuation body carried by the outer shaft,
a controller carried by the handle assembly and connected to the first actuation body for selectively prompting delivery of stimulation to the first actuation device,
wherein the first actuation body is operable to deflect the outer shaft in response to the delivered stimulation; and
a stented prosthesis loaded to the delivery device in a loaded state in which the stented prosthesis is compressed over the inner shaft and retained within the capsule.

14. The system of claim 13, wherein the first actuation body is a shape memory polymer.

15. The system of claim 13, wherein the first actuation body is electrically connected to the controller.

16. (canceled)

17. The system of claim 13, wherein the first actuation body is located at a distal region of the outer shaft.

18. (canceled)

19. The system of claim 17, wherein the first actuation body has length greater than a width, and further wherein the first actuation body is arranged relative to the outer shaft such that the width extends along a circumference of the outer shaft.

20. The system of claim 19, further comprising a second actuation body carried by the outer shaft and connected to the controller.

21. The system of claim 20, wherein the second actuation body is located opposite the first actuation body relative to the circumference of the outer shaft.

22. The system of claim 21, further comprising a third actuation body and a fourth actuation body both carried by the outer shaft and connected to the controller, wherein the first-fourth actuation bodies are equidistantly spaced from one another along the circumference of the outer shaft.

23. The system of claim 22, wherein the controller includes a joy stick.

24. The system of claim 13, wherein the stented prosthesis is a stented prosthetic heart valve.
